(19) United States
(12) Patent Application Publication
(10) Pub. No.: US 2010/0049056 A1
(43) Pub. Date: Feb. 25, 2010

(54) METHOD, DEVICE, MEDICAL IMAGE ACQUISITION DEVICE FOR IMAGING AN INTERIOR OF A TURBID MEDIUM WITH DARKNESS LEVEL MEASUREMENT

(75) Inventors: Willem Peter Van Der Brug, Eindhoven (NL); Alphonsus Tarcisius Jozef Maria Schipper, Eindhoven (NL); Jan Pieter Meeuwse, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/516,368

(22) PCT Filed: Nov. 21, 2007
(86) PCT No.: PCT/IB2007/054728

§ 371 Date: May 27, 2009

(30) Foreign Application Priority Data
Nov. 28, 2006 (EP) 06124880.3

(51) Int. Cl. A61B 6/00; G06K 9/00

(52) U.S. Cl. 600/476; 382/128

(57) ABSTRACT

The invention relates to a method, device, and medical image acquisition device for imaging an interior of a turbid medium. The method comprises the following steps: accommodation of the turbid medium inside a receiving volume (5); coupling light from an irradiation light source into the receiving volume (20); detecting light emanating from the receiving volume as a result of coupling light from the irradiation light source into the receiving volume (25); reconstructing an image of an interior of the turbid medium on the basis of detected light (30). It is an object of the invention to reduce the effect of light from a light source other than the irradiation light source on image quality. According to the invention this object is realized in that the method further comprises the following steps: measuring the light level inside the receiving volume at at least one position relative to the turbid medium when no light from the irradiation light source is coupled into the receiving volume (10); presenting information relating to the light level inside the receiving volume at at least one position relative to the turbid medium when no light from the irradiation light source is coupled into the receiving volume through use of a light level indicator (15). According to the invention the device and medical image acquisition device comprise: a photodetector unit for detecting light emanating from the receiving volume when no light from the irradiation light source is coupled into the receiving volume; a light level indicator for presenting information relating to the light level inside the receiving volume when no light from the irradiation light source is coupled into the receiving volume according to the method according to the invention.

### FIELD OF THE INVENTION

The invention relates to a method of imaging an interior of a turbid medium comprising:
accommodation of the turbid medium inside a receiving volume;
coupling light from an irradiation light source into the receiving volume;
detecting light emanating from the receiving volume as a result of coupling light from the irradiation light source into the receiving volume;
reconstructing an image of an interior of the turbid medium on the basis of detected light.

The invention also relates to a device for imaging an interior of a turbid medium comprising:
a receiving volume for accommodating the turbid medium;
an irradiation light source for generating light to be coupled into the receiving volume;
a photodetector unit for detecting light emanating from the receiving volume as a result of coupling light from the irradiation light source into the receiving volume;
an image reconstruction unit for reconstructing an image of an interior of the turbid medium on the basis of detected light.

The invention also relates to a medical image acquisition device comprising:
a receiving volume for accommodating the turbid medium;
an irradiation light source for generating light to be coupled into the receiving volume;
a photodetector unit for detecting light emanating from the receiving volume as a result of coupling light from the irradiation light source into the receiving volume;
an image reconstruction unit for reconstructing an image of an interior of the turbid medium on the basis of detected light.

### BACKGROUND OF THE INVENTION

An embodiment of a method and device of this kind is known from U.S. Pat. No. 6,327,488B1. The described method and device can be used for imaging an interior of a turbid medium, such as biological tissue. In medical diagnostics, the method and device may be used for imaging, for instance, an interior of a female breast. The receiving volume receives a turbid medium, such as a breast. Next, light from the irradiation light source is coupled into the receiving volume. This light is chosen such that it can propagate through the turbid medium. Typically the light has a wavelength within the range of 400 to 1400 nm. Light emanating from the receiving volume as a result of coupling light from the irradiation light source into the receiving volume is detected through use of the photodetector unit. Based on the detected light an image of an interior of the turbid medium, for instance a female breast, is then reconstructed.

It is a characteristic of the known method and device that a measurement as described above is sensitive to light from other light sources than the irradiation light source. Such a light source other than the irradiation light source may, for instance, be a light source used to illuminate the surroundings of the device for imaging an interior of a turbid medium. If light from a light source other than the irradiation light source enters the receiving volume and if at least a part of this light is subsequently detected through use of the photodetector unit, the quality of a reconstructed image will be negatively affected.

### SUMMARY OF THE INVENTION

It is an object of the invention to reduce the effect of light from a light source other than the irradiation light source on image quality. According to the invention this object is realized in that the method further comprises the following steps:
measuring the light level inside the receiving volume at at least one position relative to the turbid medium when no light from the irradiation light source is coupled into the receiving volume;
presenting information relating to the light level inside the receiving volume at at least one position relative to the turbid medium when no light from the irradiation light source is coupled into the receiving volume through use of a light level indicator.

The invention is based on the recognition that measuring the light level inside the receiving volume when no light from the irradiation light source is coupled into the receiving volume enables determination of whether this light is acceptable or not compared to the intensity of light emanating from the receiving volume as a result of coupling light from the irradiation light source into the receiving volume. If the light level inside the receiving volume when no light from the irradiation light source is coupled into the receiving volume is below a predetermined threshold, the effect of light from a light source other than the irradiation light source on the quality of a reconstructed image will be acceptable. If the light level inside the receiving volume when no light from the irradiation light source is coupled into the receiving volume exceeds the predetermined threshold, this may be a reason to terminate the scanning of the turbid medium under investigation. If a scan is terminated automatically, the light level indicator will inform an operator of a device for imaging an interior of a turbid medium according to the method according to the invention about the reason of the termination. If scan is not terminated automatically, the light level indicator will present an operator of a device for imaging an interior of a turbid medium according to the method according to the invention with information relating to the light level inside the receiving volume when no light from the irradiation light source is coupled into the receiving volume on the basis of which the operator can decide whether or not to terminate the scan. After a scan has been terminated, measures aimed at reducing the light level inside the receiving volume resulting from light from the light source other than the irradiation light source can be taken and a new scan initiated.

An embodiment of the method according to the invention is characterized in that in the step of measuring the light level inside the receiving volume when no light from the irradiation light source is coupled into the receiving volume, the light level inside the receiving volume is measured at at least two positions relative to the turbid medium. This embodiment has the advantage that measuring the light level inside the receiving volume when no light from the irradiation light source is coupled into the receiving volume and multiple positions relative to the turbid medium enables location of the area or areas of the receiving volume where light from a light source other than the irradiation light source enters the receiving volume. With information about where light from a light source other than the irradiation light source enters the receiving volume, location specific measures can be taken to reduce the amount of light from a light source other than the irradiation light source entering the receiving volume.

A further embodiment of the method according to the invention is characterized in that in the step of presenting information through use of a light level indicator, the information is presented visually. This embodiment has the advantage information presented visually through the use of a light level indicator can be easily accessed at any desired moment by an operator of a device for imaging an interior of a turbid medium according to the method according to the invention. Visually presented information further has the advantage that the information can be easily presented relative to a representation of the receiving volume so that the operator has an understanding of where light from a light source other than the irradiation light source is entering the receiving volume.

A further embodiment of the method according to the invention is characterized in that in step of presenting information through use of a light level indicator, the information is presented on a display. This embodiment has the advantage that it provides an easy and flexible means for presenting information in various, possibly complex ways. A display can be used to show one or more representations of the receiving volume with each representation ranging from simple to relatively complex, while at the same time allowing use of, for instance, grayscales or color scales to represent information relating to the light level inside the receiving volume.

A further embodiment of the method according to the invention is characterized in that in the step of presenting information through use of a light level indicator, the light level indicator indicates the light level inside the receiving volume when no light from the irradiation light source is coupled into the receiving volume as being either acceptable or unacceptable. This embodiment has the advantage that it provides a clear, unambiguous indication of the light level inside the receiving volume when no light from the irradiation light source is coupled into the receiving volume. Because of its simplicity, this embodiment is also easy to implement.

A further embodiment of the method according to the invention is characterized in that in the step of presenting information through use of a light level indicator, the information gives an indication of the absolute light level inside the receiving volume when no light from the irradiation light source is coupled into the receiving volume. In this embodiment, the light level inside the receiving volume can be indicated by, for instance, a single number. On the basis of this number and using his experience, an operator of a device for imaging an interior of a turbid medium according to the method according to the invention can make a judgment whether the light level inside the receiving volume is acceptable or not and whether or not to proceed with the imaging process.

A further embodiment of the method according to the invention is characterized in that in the step of presenting information through use of a light level indicator, the information is presented using a bar, with a length of the bar depending on the light level inside the receiving volume when no light from the irradiation light source is coupled into the receiving volume. This embodiment has the advantage that it provides an easy means for communicating not only whether or not the light level inside the receiving volume is acceptable or not, but also to which degree this is a case.

A further embodiment of the method according to the invention is characterized in that in the step of presenting information through use of a light level indicator, the information is presented relative to a representation of the receiving volume. This embodiment has the advantage that an operator of a device for imaging an interior of a turbid medium according to the method according to the invention can be informed about where light from a light source other than the irradiation light source is entering the receiving volume.

A further embodiment of the method according to the invention is characterized in that in the step of presenting information through use of a light level indicator, the information is presented using a grayscale. This embodiment has the advantage that it provides a means for communicating not only whether or not the light level inside the receiving volume is acceptable or not, but also to which degree this is the case.

A further embodiment of the method according to the invention is characterized in that in the step of presenting information through use of a light level indicator, information is presented using a color scale. This embodiment has the advantage that it provides a means for communicating not only whether or not the light level inside the receiving volume is acceptable or not, but also to which degree this is the case. Different colors may be used to indicate whether the light level inside the receiving volume is acceptable or not, with different use of one color indicating to which degree this is a case.

The object of the invention is further realized in that with a device for imaging an interior of a turbid medium comprising:
a receiving volume for accommodating the turbid medium;
an irradiation light source for generating light to be coupled into the receiving volume;
a photodetector unit for detecting light emanating from the receiving volume as a result of coupling light from the irradiation light source into the receiving volume;
an image reconstruction unit for reconstructing an image of an interior of the turbid medium on the basis of detected light,
   characterized in that

the device further comprises:
a further photodetector unit for detecting light emanating from the receiving volume when no light from the irradiation light source is coupled into the receiving volume;
a light level indicator for presenting information relating to the light level inside the receiving volume when no light from the irradiation light source is coupled into the receiving volume according to the method as claimed in any one of the embodiments of the method according to the invention.

An embodiment of the device according to the invention is characterized in that the photodetector unit and the further photodetector unit are comprised in a single photodetector unit. This embodiment has the advantage that no additional photodetector unit is required to determine the light level inside the receiving volume when no light from the irradiation light source is coupled into the receiving volume.

The object of the invention is further realized with a medical image acquisition device comprising:
a receiving volume for accommodating the turbid medium;
an irradiation light source for generating light to be coupled into the receiving volume;
a photodetector unit for detecting light emanating from the receiving volume as a result of coupling light from the irradiation light source into the receiving volume;
an image reconstruction unit for reconstructing an image of an interior of the turbid medium on the basis of detected light,
   characterized in that

the medical image acquisition device further comprises:
a further photodetector unit for detecting light emanating from the receiving volume when no light from the irradiation light source is coupled into the receiving volume;
a light level indicator for presenting information relating to the light level inside the receiving volume when no light from the irradiation light source is coupled into the receiving volume according to the method as claimed in any one of the embodiments of the method according to the invention.

An embodiment of the medical image acquisition device according to the invention is characterized in that the photodetector unit and the further photodetector unit are comprised in a single photodetector unit. This embodiment has the advantage that no additional photodetector unit is required to determine the light level inside the receiving volume when no light from the irradiation light source is coupled into the receiving volume.

### BRIEF DESCRIPTION OF THE DRAWINGS

These and other aspects of the invention will be further elucidated and described with reference to the drawings, in which:

FIG. 1 schematically shows an embodiment of the method according to the invention;

FIG. 2 schematically shows an embodiment of a visual light level indicator according to the invention using a bar the length of which indicates the light level inside the receiving volume when no light from the irradiation light source is coupled into the receiving volume;

FIG. 3 schematically shows a further embodiment of a visual light level indicator according to the invention with information relating to the light level inside the receiving volume being presented relative to a representation of the receiving volume;

FIG. 4 schematically shows a further embodiment of a visual light level indicator according to the invention with information relating to the light level inside the receiving volume being presented relative to a representation of the receiving volume using a grayscale.

FIG. 5 schematically shows a further embodiment of a visual light level indicator according to the invention with information relating to the light level inside the receiving volume being presented relative to a representation of the receiving volume on a display.

FIG. 6 schematically shows an embodiment of a device for imaging an interior of a turbid medium according to the method according to the invention;

FIG. 7 schematically shows an embodiment of a medical image acquisition device according to the method according to the invention.

### DETAILED DESCRIPTION OF EMBODIMENTS

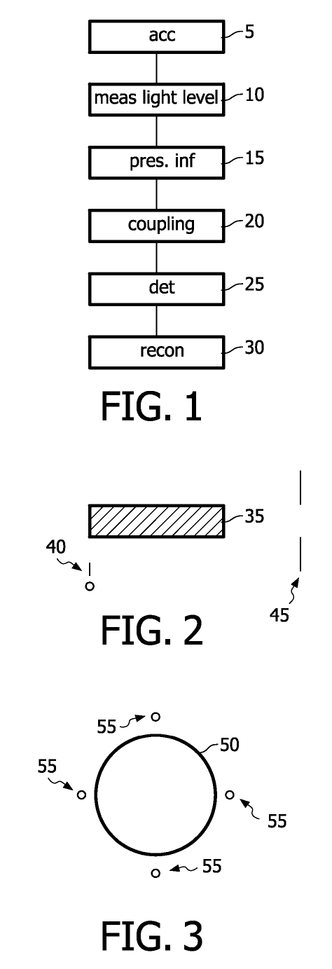

FIG. 1 schematically shows an embodiment of the method according to the invention. In step 5 a turbid medium is accommodated inside a receiving volume. Next, in step 10 and according to the invention, the light level inside the receiving volume is measured at at least one position relative to the turbid medium when no light from the irradiation light source is coupled into the receiving volume. Information relating to the light level inside the receiving volume at the at least one position relative to the turbid medium when no light from the irradiation light source is coupled into the receiving volume is then presented in step 15. The information is presented through use of a light level indicator and can be presented in various ways. One way is to use an acoustic signal to inform an operator of a device according to the method according to the invention about whether the light level inside the receiving volume and no light from the irradiation light source is coupled into the receiving volume is acceptable or not. The acoustic signal can have different tones, with one tone being used to signal that the light level inside the receiving volume is acceptable and another tone being used to signal that this light level is unacceptable. Alternatively, one or more voice signals can be used. Another way is to present the information visually. One way to do this is to generate a simple acceptable/unacceptable signal. This can be done, for instance, through use of a green light for indicating that the light level inside the receiving volume when no light from the irradiation light source is coupled into the receiving volume is acceptable and use of a red light indicating that this light level is unacceptable. Alternatively, different signs can be displayed on a screen to indicate that the light level inside the receiving volume is either acceptable or unacceptable. A number of other ways to present the information visually will be discussed further in relation to FIGS. 2-5. Depending on whether the light level inside the receiving volume when no light from the irradiation light source is coupled into the receiving volume is acceptable or not, the imaging process of the turbid medium can proceed or be terminated. The decision to either proceed with or terminate the imaging process can be made automatically or by the operator of a device according to the method according to the invention. In the latter case, the operator will make his decision based on the presented information relating to the light level inside the receiving volume when no light from the irradiation light source is coupled into the receiving volume. In medical diagnostics, a device for imaging an interior of a turbid medium according to the method according to the invention may be used for imaging an interior of, for instance, a female breast. Such a device may comprise a receiving volume bounded by a wall in the shape of a cup having one open side. The open side allows a patient's breast to be accommodated inside the receiving volume. If the light level inside the receiving volume when no light from the irradiation light source is coupled into the receiving volume turns out to be unacceptably high, this means that there is at least one position on the boundary of the open side at which light from a light source other than the irradiation light source is able to enter the receiving volume. Once presented with the information that the light level inside the receiving volume is unacceptably high, an operator of the device can then reposition the patient in order to reduce the amount of light from a light source other than the irradiation light source entering the receiving volume. The operator repositions the patient until he is informed by the light level indicator that the light level inside the receiving volume when no light from the irradiation light source is coupled into the receiving volume has become acceptable for the imaging process to proceed. Especially if the light level inside the receiving volume when no light from the irradiation light is coupled into the receiving volume is measured at a plurality of positions relative to the turbid medium, and if information relating to the light level inside the receiving volume obtained at the plurality of positions is presented relative to a representation of the receiving volume, the operator will have an understanding of where light from a light source other than the irradiation light source is able to enter the receiving volume. The operator can then reposition the patient accordingly. Once the light level inside the receiving volume when no light from the irradiation light source is coupled into the receiving volume has become acceptable, the process of imaging an interior of the turbid medium can proceed. To this end, light from the irradiation light source is coupled into the receiving volume. This is done in step 20. Next, in step 25, light emanating from the receiving volume as a result of coupling light from the irradiation light source into the receiving volume is detected through use of a photodetector unit. Based on the detected light, an image of an interior of the turbid medium is subsequently reconstructed in step 30. Performing step 10 of measuring the light level inside the receiving volume when no light from the irradiation light source is coupled into the receiving volume and step 15 of presenting information relating to the light level inside the receiving volume when no light from the irradiation light source is coupled into the receiving volume is not limited to the period prior to scanning the turbid medium inside the receiving volume. Both step 10 and step 15 may also be performed during the scanning of a turbid medium. In a device for imaging an interior of a turbid medium according to the method according to the invention, the turbid medium may, for instance, be irradiated with light from the irradiation light source by coupling light from the irradiation light source into the receiving volume using an entrance position for light successively chosen from a plurality of entrance positions for light comprised in the receiving volume. While switching the irradiation light source from one entrance position for light to the next, no light from the irradiation light source is coupled into the receiving volume. During that time interval, that is during the process of scanning the turbid medium, the light level inside the receiving volume may be measured to determine whether an unacceptable amount of light from a light source other than the irradiation light source is able to enter the receiving volume.

FIG. 2 schematically shows an embodiment of a visual light level indicator according to the invention using a bar the length of which indicates the light level inside the receiving volume when no light from the irradiation light source is coupled into the receiving volume. In FIG. 2 the rectangle marked 35 indicates a bar the length of which indicates the light level inside the receiving volume when no light from the irradiation light source is coupled into the receiving volume. If this light level lies within the lower detection limit of the means for measuring the light level inside the receiving volume when no light from the irradiation light source is coupled into the receiving volume, the bar 35 will have substantially zero length. The position at which the bar 35 has substantially zero length is indicated by the arrow marked 40. As the light level inside the receiving volume when no light from the irradiation light source is coupled into the receiving volume increases, and the length of the bar 35 increases accordingly starting from the position marked 40, there will come a point at which this light level is no longer acceptable. In FIG. 2 this threshold point has been marked 45. In addition to the length of the bar 35 depending on the light level inside the receiving volume when no light from the irradiation light source is coupled into the receiving volume, the bar 35 may be grayscale-coded or color-coded to indicate the light level inside the receiving volume. The use of a color-code has the advantage that a clear distinction can be made between acceptable and unacceptable light levels through use of different colors for acceptable and unacceptable light levels. The degree to which a specific light level is acceptable or unacceptable may then be indicated through use of different hues of a single color. Clearly, the bar 35 need not be continuous, but may, as one possible alternative, comprise a series of lights, with the number of activated lights, and possibly there are color, being used to indicate the light level inside the receiving volume when no light from the irradiation light source is coupled into the receiving volume.

FIG. 3 schematically shows a further embodiment of a visual light level indicator according to the invention with information relating to the light level inside the receiving volume being presented relative to a representation of the receiving volume. As was discussed in relation to FIG. 1, the receiving volume for accommodating the turbid medium may be bounded by a wall. In medical diagnostics, where a device for imaging an interior of a turbid medium according to the method according to the invention may be used for imaging, for instance, a female breast, this wall can have the shape of a cup with one open side. Especially if the light level inside the receiving volume when no light from the irradiation light source is coupled into the receiving volume is measured at a plurality of positions relative to the turbid medium, presenting information relating to this light level relative to a representation of the receiving volume is advantageous. The advantage lies in the fact that an operator of a device for imaging an interior of a turbid medium, for instance, a female breast, that uses the method according to the invention is informed about where relative to the turbid medium the light level inside the receiving volume is acceptable or unacceptable. Using this information the operator can reposition the patient to reduce the amount of light from a light source other than the irradiation light source that is able to enter the receiving volume. FIG. 3 schematically shows one way to present information relating to the light level inside the receiving volume relative to a representation of the receiving volume. The circle 50 represents the perimeter of the open side of a cup-shaped wall bounding a receiving volume. This representation of the receiving volume may be displayed on screen or applied to a visible portion of a device for imaging an interior of a turbid medium according to the method according to the invention. Lights 55 distributed along the circle 50 representing the receiving volume are used to indicate whether the light level inside the receiving volume when no light from the irradiation light source is coupled into the receiving volume is acceptable or not. The color of each of the lights 55 indicates whether this light level is acceptable or not in the part of the receiving volume represented by each of the lights 55. Clearly, the number of lights 55 distributed along the representation of the receiving volume is not limited to the number of lights shown in FIG. 3.

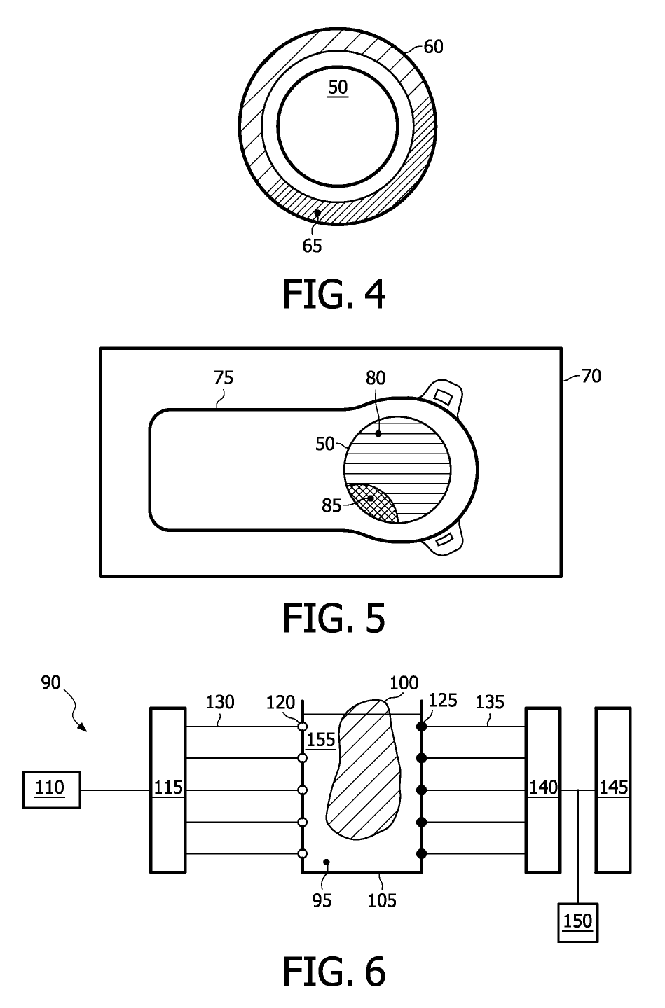

FIG. 4 schematically shows a further embodiment of a visual light level indicator according to the invention with information relating to the light level inside the receiving volume being presented relative to a representation of the receiving volume using a grayscale. Just as in FIG. 3, the circle 50 represents the perimeter of the open side of a cup-shaped wall bounding a receiving volume. However, instead of lights 55 a ring-shaped display 60 surrounds the circle 55. A grayscale can be used to present information relating to the light level inside the receiving volume when no light from the irradiation light source is coupled into the receiving volume on the display 60. Different shades of gray then indicate different light levels inside the receiving volume. In FIG. 4 different shades of gray have been indicated by differently shaded areas, with, for instance, the relatively densely shaded area 65 indicating that an unacceptable amount of light from a light source other than the irradiation light source is entering the receiving volume in that area. The less densely shaded area then indicates an area in which the light level is acceptable. Alternatively, a color scheme analogous to the color scheme discussed in relation to the bar 35 in FIG. 2 can be used to present information on the display 60.

FIG. 5 schematically shows a further embodiment of a visual light level indicator according to the invention with information relating to the light level inside the receiving volume being presented relative to a representation of the receiving volume on a display. The display 70 shows a representation of a device 75 for imaging an interior of a turbid medium, such as a female breast. The representation of the device 75 comprises a circle 50 representing a boundary of the receiving volume comprised in the device 75. In FIG. 5 two areas, area 80 and area 85, had been indicated inside a representation of the receiving volume. Area 80 represents a part of the receiving volume in which the light level when no light from the irradiation light source is coupled into the receiving volume. Area 85 represents a part of the receiving volume in which the light level when no light from the irradiation light source is coupled into the receiving volume is unacceptable. In FIG. 5 area 80 and area 85 can be distinguished from each other because they are shaded differently. Alternatively, a grayscale or a color scale, analogous to the color scale discussed in relation to the bar 35 in FIG. 2, can be used to distinguish between different areas in the representation of the receiving volume.

FIG. 6 schematically shows an embodiment of a device for imaging an interior of a turbid medium according to the method according to the invention. The device 90 comprises a receiving volume 95 for receiving a turbid medium 100. The receiving volume 95 is limited by a wall 105. Light from an irradiation light source 110 is coupled into the receiving volume 95 through use of the selection unit 115. The selection unit 115 is used to successively select an entrance position for light from the plurality of entrance positions for light 120. Light emanating from the receiving volume 95 as a result of coupling light from the irradiation light source 110 into the receiving volume 95 exits the receiving volume 95 using the plurality of exit positions for light 125. Light emanating from the receiving volume 95 is detected through use of the photodetector unit 140. The plurality of entrance positions for light 120 are optically coupled to the selection unit 115 using light guides 130. The plurality of exit positions for light 125 are optically coupled to the photodetector unit 140 using light guides 125. Image reconstruction unit 145 is used to reconstruct an image of an interior of the turbid medium 100 based on the detected light. According to the invention, the photodetector unit 140 is further used for determining the light level inside the receiving volume 95 when no light from the irradiation light source 110 is coupled into the receiving volume 95. To present information relating to the light level inside the receiving volume 95 when no light from the irradiation light source 110 is coupled into the receiving volume 95 the device 90 further comprises a light level indicator 150. Inside the receiving volume 95, the turbid medium 100 may be surrounded by a matching medium 155. The matching medium 155 has optical properties, such as an absorption coefficient, similar to those of the turbid medium 100. In this way, boundary effects stemming from coupling light from the irradiation light source 110 into and out of the turbid medium 100 are reduced and optical short-circuits around the turbid medium 100 prevented.

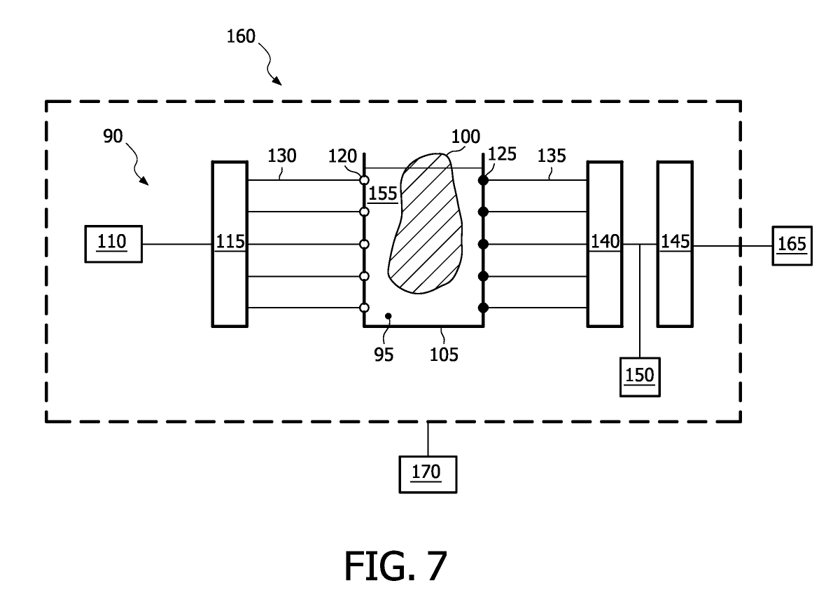

FIG. 7 schematically shows an embodiment of a medical image acquisition device according to the method according to the invention. The medical image acquisition device 160 comprises the elements of the device 90 shown in FIG. 6 as indicated by the dashed square. Additionally, the medical image acquisition device 160 further comprises a screen 165 for displaying a reconstructed image of an interior of the turbid medium 100 and an operator interface 170, such as a keyboard, allowing an operator to interact with the medical image acquisition device 160.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. In the system claims enumerating several means, several of these means can be embodied by one and the same item of computer readable software or hardware. The mere fact that certain measures are recited in mutually different dependent claims does not indicate that a combination of these measures cannot be used to advantage.

1. A method of imaging an interior of a turbid medium comprising:
accommodation of the turbid medium inside a receiving volume;
coupling light from an irradiation light source into the receiving volume;
detecting light emanating from the receiving volume as a result of coupling light from the irradiation light source into the receiving volume;
reconstructing an image of an interior of the turbid medium on the basis of detected light,
characterized in that
the method further comprises the following steps:
measuring the light level inside the receiving volume at at least one position relative to the turbid medium when no light from the irradiation light source is coupled into the receiving volume;
presenting information relating to the light level inside the receiving volume at at least one position relative to the turbid medium when no light from the irradiation light source is coupled into the receiving volume through use of a light level indicator.

2. A method as claimed in claim 1, wherein in the step of measuring the light level inside the receiving volume when no light from the irradiation light source is coupled into the receiving volume, the light level inside the receiving volume is measured at at least two positions relative to the turbid medium.

3. A method as claimed in claim 1, wherein in the step of presenting information through use of a light level indicator, the information is presented visually.

4. A method as claimed in claim 3, wherein in the step of presenting information through use of a light level indicator, the information is presented on a display.

5. A method as claimed in claim 3, wherein in the step of presenting information through use of a light level indicator, the light level indicator indicates the light level inside the receiving volume when no light from the irradiation light source is coupled into the receiving volume as being either acceptable or unacceptable.

6. A method as claimed in claim 3, wherein in the step of presenting information through use of a light level indicator, the information gives an indication of the absolute light level inside the receiving volume when no light from the irradiation light source is coupled into the receiving volume.

7. A method as claimed in claim 3, wherein in the step of presenting information through use of a light level indicator, the information is presented using a bar, with a length of the bar depending on the light level inside the receiving volume when no light from the irradiation light source is coupled into the receiving volume.

8. A method as claimed in claim 3, wherein in the step of presenting information through use of a light level indicator, the information is presented relative to a representation of the receiving volume.

9. A method as claimed in claim 3, wherein in the step of presenting information through use of a light level indicator, the information is presented using a grayscale.

10 A method as claimed in claim 3, wherein in the step of presenting information through use of a light level indicator, information is presented using a color scale.

11. A device for imaging an interior of a turbid medium comprising:
a receiving volume for accommodating the turbid medium;
an irradiation light source for generating light to be coupled into the receiving volume;
a photodetector unit for detecting light emanating from the receiving volume as a result of coupling light from the irradiation light source into the receiving volume;
an image reconstruction unit for reconstructing an image of an interior of the turbid medium on the basis of detected light,
characterized in that
the device further comprises:
a further photodetector unit for detecting light emanating from the receiving volume when no light from the irradiation light source is coupled into the receiving volume;
a light level indicator for presenting information relating to the light level inside the receiving volume when no light from the irradiation light source is coupled into the receiving volume according to the method as claimed in claim 1.

12. A device as claimed in claim 11, wherein the photodetector unit and the further photodetector unit are comprised in a single photodetector unit.

13. A medical image acquisition device comprising:
a receiving volume for accommodating the turbid medium;
an irradiation light source for generating light to be coupled into the receiving volume;
a photodetector unit for detecting light emanating from the receiving volume as a result of coupling light from the irradiation light source into the receiving volume;
an image reconstruction unit for reconstructing an image of an interior of the turbid medium on the basis of detected light,
characterized in that
the medical image acquisition device further comprises:
a further photodetector unit for detecting light emanating from the receiving volume when no light from the irradiation light source is coupled into the receiving volume;
a light level indicator for presenting information relating to the light level inside the receiving volume when no light from the irradiation light source is coupled into the receiving volume according to the method as claimed in any one of the embodiments of the method according to the invention.

14. A medical image acquisition device as claimed in claim 13, wherein the photodetector unit and the further photodetector unit are comprised in a single photodetector unit.
